(19) United States
(12) Patent Application Publication
(10) Pub. No.: US 2022/0192890 A1
(43) Pub. Date: Jun. 23, 2022

(54) WOUND TREATMENT SYSTEM AND METHOD

(71) Applicant: Zam Research LLC, Overland Park, KS (US)

(72) Inventors: Jacob G. Hodge, Merriam, KS (US); David S. Zamierowski, Overland Park, KS (US); Adam J. Mellott, Olathe, KS (US)

(21) Appl. No.: 17/555,044

(22) Filed: Dec. 17, 2021

Related U.S. Application Data
(60) Provisional application No. 63/127,364, filed on Dec. 18, 2020.

(51) Int. Cl. A61F 13/02; A61L 15/42; A61L 15/22; A61L 15/60; A61M 1/00

(52) U.S. Cl. CPC A61F 13/0216; A61L 15/425; A61M 1/90; A61L 15/60; A61L 15/225

(57) ABSTRACT

A system for treating open wounds includes a foam material configured for pouring, spraying, injecting or spreading on a wound bed. The foam material can comprise a base component and a curing component, which can be pre-mixed before application, or mixed in situ as the components are being applied to the wound site. A third component can comprise a sacrificial porogen. Foam can be placed in the wound bed as a wound liner on the wound surfaces. An additional foam insulation can provide a foam filler partially contained by the wound liner and generally flush with a patient's epidermis. A method of treating open wounds includes the steps of applying the wound liner and filler components. An optional step comprises covering the wound liner with a semi-permeable (breathable) membrane and mounting inlet and outlet ports thereon for introducing healing compositions as input, and extracting wound exudates as output.

### CROSS-REFERENCE TO RELATED APPLICATION

This application claims priority in U.S. Provisional Patent Application No. 63/127,364 Filed Dec. 18, 2020, which is incorporated herein by reference.

### BACKGROUND OF THE INVENTION

### 1. Field of the Invention

The present invention relates generally to tissue engineering for wound repair and regeneration methods, and more specifically to the use of a multi-solution system containing bioactive factors that, once combined, form a gel or expanding foam that forms a wound liner, a wound filler, or a scaffold or structure between elements to be joined or healed which supports regenerative tissue engineering.

### 2. Description of the Related Art

Tissue wound healing involves a complex and intricate set of interrelated, systematic events. The dynamic nature of these cascading events can go awry based on any one of many aberrant processes which can result in failure of proper wound healing and lead to long-term, pathological problems. Failure of wounds to heal properly after trauma, surgery, or acute or chronic diseases affects millions of people every year, with chronic wounds costing patients tens of billions of dollars collectively.

Complex wounds involving tissue loss and/or damage to skin, cartilage, bone, muscle, nerves, or blood vessels often require extensive surgical correction and long-term treatment modalities to attempt to restore normal anatomical structure and function. Full recovery of anatomical homeostasis can be hindered by limitations of the tissue healing process and therapeutic treatments. Complex wounds are more likely to result in chronic non-healing wounds than anatomically simpler wounds, and chronic non-healing wounds have a direct effect on increased morbidity and mortality in patients. Chronic wounds are prone to infection, which may progress to sepsis and death. Even if they heal, the prolonged process can result in disfigurement, loss of function, extensive scarring, and other long term sequalae. Ultimately, lack of proper wound healing in patients can lead to systemic and chronic ailments that reduce tissue structure and function and lead to a decrease in patient quality of life. Wound dressings are often considered the standard of care for many wounds, both acute and chronic, and have evolved over the years to improve patient outcomes.

The prior art includes technologies and methodologies for use of a gelatinous or foam material inserted in or on a wound to promote tissue genesis and improve wound healing, with or without the application of a pressure gradient. Open-cell, reticulated porous foam inserts are commonly used with the application of pressure gradients (i.e. negative/vacuum pressure or positive pressure) as a means to accelerate wound closure and fluid egress for surgical site wound prep, traumatic and acute wounds, ulcerative or chronic wounds, and to improve efficacy of skin grafting procedures. Small cell or closed cell foams, usually used for re-epithelialization of wounds, have also been used with pressure gradients (usually negative pressure).

The use of a porous foam wound insert with a semipermeable adhesive film connected to a vacuum source is often termed negative pressure wound therapy (NPWT). NPWT has demonstrated clinical efficacy by its ability to promote blood flow, improve the removal of fluid and edema, stimulate mechanotransduction signaling and cellular proliferation, stabilize the wound reducing microshear and augment the overall healing process. Such NPWT benefits have improved outcomes for many patients, including those afflicted with chronic and non-healing wounds.

Solution-based systems previously have been used as therapeutic options for wound care. They work by forming a gelatinous dressing or hemostatic agent, such as hydrogel-based dressings or fibrin glue. Hydrogels work by fabricating a hydrated three-dimensional (3D) gel made with biological or polymer-based compounds within a highly saturated substrate. These are often greater than 90% water by weight and can be premade and applied to a wound as a dressing to provide a moist environment and promote wound healing. Hydrogels frequently contain extracellular matrix components such as collagen, fibrin, or hyaluronic acid and are often used in conjunction with another polymer or polysaccharide, such as polyethylene glycol or chitosan, respectively.

Fibrin glue was initially used in clinical practice as a hemostatic agent to seal wounds and prevent exsanguination. More recently fibrin-based glues have been investigated for their ability to deliver bioactive components or stem cells for tissue engineering applications to promote tissue genesis. Hydrogels can be used as standalone wound dressings. Both of these types of agents have demonstrated efficacy in promoting wound healing by stimulating extracellular matrix deposition and angiogenesis.

Closed-wound injuries under intact skin, or internal injuries, include bone fractures and soft tissue ruptures and tears. Conventional therapeutic approaches include reduction, splinting and casting. In cases where aligning fractured bone segments and maintaining reduction are difficult, care providers have employed open reduction and internal fixation (ORIF) procedures. Through an incision, hardware can be applied to or inserted into bony segments to achieve alignment and firm fixation. These include metals in the form of screws, plates (with or without compression), rods, and bone segment replacements or prosthetics, as in the case of fixation of a hip fracture with a new prosthetic femoral head, and these materials include plastics and absorbable materials. However, this form of tissue repair can require surgery, which can further damage tissue. Repairing damaged soft tissue and bone can necessitate the permanent placement of fixation devices, such as screws, anchors and plates, that remain in the body.

Though these prior wound treatments have demonstrated clinical efficacy for many patients, several limitations still remain with NPWT and reticulated open-cell foam (ROCF) dressings. The limitations of NWPT include tissue enmeshing in the ROCF and the lack of ability to effectively tailor a custom foam insert for different shapes and sizes of wounds.

Tissue enmeshing presents a problem with current open-cell foam inserts used for negative pressure therapy because these foams must be removed every 1-3 days, and newly forming tissue grows into the reticulated porous foam, resulting in removal of this tissue during dressing changes. This tissue ingrowth is often attached to, or anchored with, newly formed tissue within the wound bed as well, resulting in removal of newly formed, healthy tissue and irritation of the wound site. This mechanical irritation often results in some form of an inflammatory response. Persistent inflammation is a known cause of chronic non-healing wounds. Thus, repetitive removal of dressings with enmeshed tissue can potentially lead to persistent irritation of a wound site and cause a delay in proper healing, as well as pain and discomfort for the patient.

Additionally, tissue enmeshing can provide seed points for bacteria to adhere to and propagate within a wound bed, and constant dressing changing increases the opportunity for introducing new bacteria, since, by definition, an open wound cannot be sterile. Therefore, minimizing the amount of tissue ingrowth into foam and decreasing the number of dressing changes required would offer a new way to handle dressings in open wounds, remedy some of the undesirable factors associated with prior art systems, and improve overall wound healing.

The lack of customization of foam inserts for wounds is also a major obstacle. Current technology requires one to hand cut preformed porous, open-cell foam blocks into a desired shape and then insert the cut foam into a wound. This makes it very difficult to mimic the shape, contour, and size of many wounds, especially wounds over joint articulations, complex bone architecture of the face, and complex wound shapes associated with traumatic wounds. Additionally, the range of materials approved for fabricating foam inserts for wound treatment is limited. Conforming foam materials to irregularly-shaped wounds can be time-consuming and challenging.

Surface contact with the wound is inherently desired to optimize the function of these dressings, and wounds with irregular sides, undermining, tunneling, and clefting make manual preparation of inserts technically difficult. Creating the complex shape of a wound by utilizing small blocks or pieces of the foam material, either provided by the manufacturer or manually fashioned on site, has been utilized and has dramatically increased the number of overlooked and retained foreign bodies in these wounds.

Wounds have various depths, tissues, and environments. Therefore, it would be highly beneficial to be able to easily modify the foam inserts used for these wounds to maximize the clinical efficacy of negative pressure wound systems and wound dressings in general.

Currently, gelatinous wound dressings are used as a dressing to fill, cover, and/or seal a wound. Though effective in what they are currently used for, gelatinous wound dressings can be modified and used in a different manner as a wound liner and/or foam insert during pressure gradient therapy. Current gels generally are not mechanically robust enough to withstand pressure gradient therapy and often must be used with an additional dressing to secure the gel dressing in place. Moreover, currently utilized, pre-formed gel dressings are flat and difficult to use in complex, deep, irregular wounds.

Gel pastes, on the other hand, can be difficult to apply as thin films on wound walls. Hydrogel dressings are highly saturated with water such that the wound can become overhydrated. Fibrin glue and other wound sealants are typically not used as dressings, but some wound dressings incorporate fibrin and other extracellular matrix compounds to enhance the wound healing process. When used as a sealant to bind tissue, they tend to trap exudative and edematous fluids and thus impair wound healing. Therefore, though these gelatinous wound therapies have been shown to promote vital wound healing processes, they still lack in their ability to maintain dimensional stability on their own and help promote fluid egress from the wound site.

Treatment of closed wound injuries could be improved if the need for open surgical correction and fixation could be reduced. What is desired is a pourable material to be utilized in situations having separated bone and tissues, not by open incision as described above, but by injection as a glue-like or binding agent that can provide structural support and dimensional stability to the injury, while also serving as a temporary or permanent scaffold for cells to migrate within and proliferate to form new tissue.

Such an approach to fixation would have several beneficial advantages. Experience with prosthetic joint replacements shows that reticular or trabecular formation of material with specific pore sizes allows for bone tissue ingrowth into prosthetics and firmer fixation to bone. Since injectable material can be formed into a foam or mesh liner in situ and made reticular, it could firmly become attached to respective bone fragments by ingrowth. Further, since a needle or tubing can be used to insert the foam or mesh material, such wound site access can also be used to add negative pressure to the system once the material is hardened in situ.

This adjunct therapy would speed the movement of tissue and osteoblast ingrowth into trabecular pattern prosthetics and across the bone fracture junction, thereby speeding healing strength and decreasing healing time. This approach would have the added benefit of potentially eliminating the need for open surgery, which can prolong healing and result in increased scar tissue formation. Such an approach would still need external fixators, a splint, or a cast for a prescribed amount of time for the bone to knit and gain strength. The advantage of negative pressure through a needle or catheter in this approach though is that callus-inducing hematoma can be drained and ingrowth can be induced into the trabecular structure of the injected foam. In theory, this could mean that patients would not need external splints, casts or fixators for as long.

Heretofore there has not been available a customizable, multi-solution system or method for wound treatment to prevent tissue enmeshing and to promote tissue genesis with the advantages and features of the present invention, including inter-tissue gels and/or foams with a modifiable porous fraction and inclusion of bioactive compounds, that can be poured, sprayed, injected or spread into a wound site with the ability to be utilized with or without a pressure gradient system.

### SUMMARY OF THE INVENTION

The present invention discloses an improved wound treatment dressing and method. The present approach circumvents the previously mentioned limitations of current gelatinous dressings and foam or mesh inserts utilized in wound care, with or without pressure gradient therapy. The present invention covers customizable and premade sets of multi-solution systems that form an amorphous gel or foam wound liner or an amorphous, expandable foam wound insert that can be poured, sprayed, injected, or spread to fill a wound bed for specific tissue applications. This approach allows for fabrication of a tailored, multi-solution set of systems with bioactive synthetic and natural compounds, including factors to promote tissue growth, cell migration, and proliferation; to improve the dimensional stability of gel or foam liners or wound inserts; and to augment porous fractions.

The porous fraction of the liners and inserts can either be closed-cell, with discontinuity of pores, or open-cell, with continuous pores. For example, various functional considerations may be factors in optimizing the closed-cell and open-cell pore sizes. Moreover, the invention is scalable to adapt to various applications. The pore sizes of the continuous pores of the open-cell foam tend to affect fluid transfer and flow functional parameters. Still further, the open-cell or closed-cell character of the foam can change over the course of a treatment procedure. For example, continuous pores in an open-cell configuration can close as healing occurs or when subject to negative pressure, resulting in a partially or fully-closed configuration.

In terms of physiologic tissue response, the pore size is a significant variable in determining whether the epithelial cells will be able to migrate beneath the material unimpeded or if the epithelial cells will be disrupted and unable to migrate because the granulation has enmeshed into the open pores. Even if the enmeshing is only one pore deep, epithelial cell migration can be compromised. The open or closed cell characteristics can affect the ability of a dressing to collapse and “firm-up” under negative pressure. Moisture evaporation rates and the ability to handle exudate are additional physiological functional criteria, which are affected by the foam materials and configurations, including open and closed cells, and pore sizes.

The pore sizes can be manipulated, and the connectivity of the pores can be altered to modulate the ability for fluid to pass through a wound liner. Small-cell systems decrease tissue enmeshing while still allowing the transport of fluid through the system. Systems with larger, open-cell configurations allow for both tissue ingrowth and fluid transport, while also being highly compactable or compressible to accommodate decreases in wound area under vacuum or negative pressure. In some embodiments, a sacrificial component is incorporated to augment the porous fraction of the liner and/or inserts. The ability to fine-tune pore size and porosity in a foam insert allows better control over complex and variable tissue responses. This is because the modulation of pore size and bulk porosity can have an overall effect on the compactability or compressibility of the foam insert. The compactability or compressibility will directly correlate to the contraction and deformability of the wounded tissue and therefore the degree of physicochemical and mechanotransduction response occurring within the damaged tissue.

The degree of swelling within the hydrogel and the subsequent pore size can be controlled to achieve optimum healing outcomes. For example, the composition of the hydrogel and the application of a rinsing solution can alter swelling and foam porosity factors, with corresponding effects on tissue responses and re-epitheliazation.

Additionally, the ability to have a variety of solutions with different premade compounds provides the capacity of tailoring gel or foam wound liners and/or foam inserts to specific wound applications. The use of a multi-solution system permits the ability for the curation rate of the wound liner and inserts to be modified to adjust for mode of application (i.e., pour, spray, inject, or spread) and for binding to another gel or foam liner(s) and/or insert(s), if desired. The wound liners can be used in conjunction with preformed foam inserts commonly used with pressure gradient therapy, with other wound dressings, or with the aforementioned expanding foam solution by using two different multi-solution systems. The expanding foam insert solutions can be used alone, with or without pressure gradient therapy, or with other wound dressings, including preformed foam inserts as a vacuum core and adhesive, semi-permeable dressings.

The use of a multi-solution system permits the user to pour, spray, inject, or spread amorphous solutions into a wound site where the solutions undergo a chemical reaction and cure to form a gel or foam that conforms to the shape and size of the wound and may or may not expand to fill the voided space within the wound. The expanding open-cell foam insert can be compacted or compressed under vacuum and decrease the wound area, and if used in conjunction with a closed-cell wound liner, tissue enmeshing will be limited. This overall approach eliminates the need to cut inserts by hand while permitting the customizability of solutions and reduction of tissue enmeshing from using a closed-cell wound liner system. The use of a solution-based system permits the manufacturer and the user to create additional solution systems for either liners or wound inserts and bind them together instead of using premade gels or foams that are made in generic shapes and sizes. Incorporation of sacrificial porogens, including solutes, gases, salts, and particles, can alter the porous fraction of the final wound liner and/or foam inserts. Specific combinations shown to be effective for particular wounds can be provided.

### BRIEF DESCRIPTION OF THE DRAWINGS

The drawings constitute a part of this specification and include exemplary embodiments of the present invention illustrating various objects and features thereof.

FIG. 1 is a cross-sectional view of an open wound, which can be treated with the present invention.

FIG. 2 shows a liner applied to the wound.

FIG. 3 shows foam applied as filler for the wound.

FIG. 4 shows an optional insert installed in the foam.

FIG. 5 shows a re-epithelialized, healing, outcome.

FIG. 6 shows a modified or alternative embodiment of the present invention configured for negative pressure wound therapy (NPWT), with a semi-permeable membrane cover and inlet and outlet ports.

FIG. 7 shows another modified or alternative embodiment of the present invention with foam applied directly to the exposed tissue in the wound bed.

### DETAILED DESCRIPTION OF THE PREFERRED EMBODIMENTS

### I. Introduction and Environment

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. For example, up, down, front, back, right, and left refer to the invention as orientated in the view being referred to. The words “inwardly” and “outwardly” refer to directions toward and away from, respectively, the geometric center of the aspect being described and designated parts thereof. Forwardly and rearwardly are generally in reference to the direction of travel, if appropriate. Additionally, anatomical terms are given their usual meanings. For example, proximal means closer to the trunk of the body, and distal means further from the trunk of the body. Said terminology will include the words specifically mentioned, derivatives thereof and words of similar meaning.

As described herein, the term foam shall be defined as any liquid or solid material having pockets of gas within the liquid or solid material, including both open-cell and closed-cell pockets. The term foam shall be interpreted broadly enough to include materials of any thickness, including thin materials such as meshes. Furthermore, the terms cure and curing shall be defined as the process of hardening a material, including but not limited to cross-linking of biological and/or synthetic components. A curing agent shall mean a substance or factor applied to a material to initiate curing of that material.

### II. Preferred Embodiments

The present invention discloses an improved system and method for wound treatment. In an exemplary embodiment, a curable, amorphous wound dressing is applied to a wound in a liquid or semi-liquid state, which dressing forms to the shape of the wound cavity and cures within the wound. Preferably, the wound dressing forms into a foam material. Embodiments include both open-cell and closed-cell dressings, and the wound dressing material components can be configured for being poured, sprayed, injected, or spread into a wound. The wound dressing can include pores which are sized for physiologic effect, i.e., large, granulation-enmeshing or small, non-enmeshing pores. Additionally, the dressing may be hydrophobic or hydrophilic.

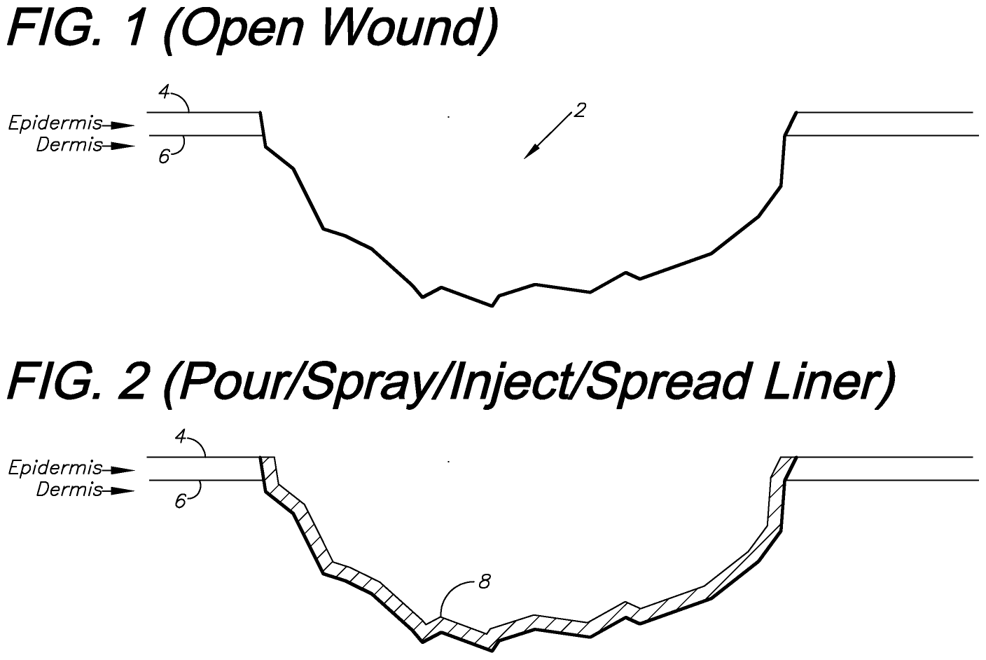

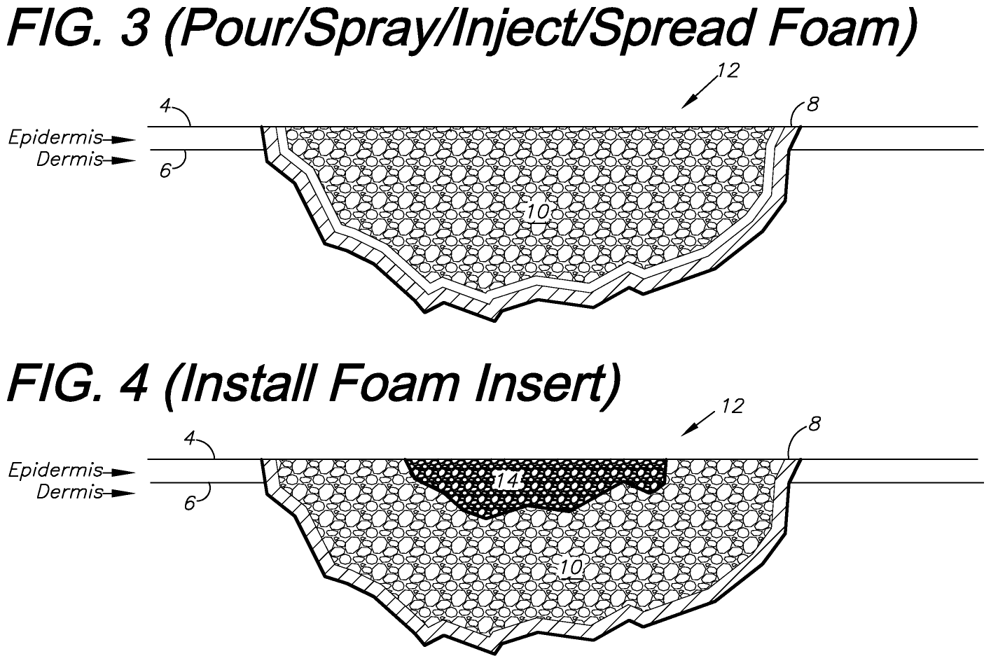

FIG. 1 shows an open wound 2 penetrating the epidermis 4 and extending into the dermis 6. Such wounds can be caused by a variety of conditions, including disease and trauma. Diabetes, impaired circulation, prolonged patient immobility and other medical conditions can exacerbate such wounds. If left untreated, the negative consequences can include infection, functional disability, limb loss and even death. FIGS. 2 and 3, respectively, show the application of a foam wound liner 8 and a foam filler 10 generally flush with the patient's epidermis 4.

Without limitation on the generality of useful materials for forming a wound dressing 12 according to the present invention, the foam liner 8 and the filler 10 can be gel and/or foam, which can comprise premade, tailored solutions. Such solutions can include first and second compounds, which are the components of the gel and/or foam liner. A third, additional compound can comprise, for example, a sacrificial porogen (e.g., bioactive factors, curing agent or adhesion protein). The third compound can be added to either the first or second compound, or it can be added separately and allowed to mix within the wound 2 bed upon application with the first and second compounds.

The third compound can consist of a mixture of multiple solutions or be separated into multiple distinct solutions. Utilizing an application system such as a dual syringe (or other modality), the first and second compounds can be kept separate until application to the wound 2. Alternatively, they can be mixed together in a single chamber and then applied, depending on the compounds used and the method of curation.

Upon application of the multi-compound to the wound 2, the solution mixture will disperse throughout the wound 2 and cure to form a wound liner layer 8. The wound liner 8 can be used as its own modality or with other wound dressings and therapies, such as the foam filler 10. FIG. 4 shows a porous foam insert 14 embedded in the foam filler 10. The insert 14 can be used to alter the performance characteristics of the dressing 12, such as directing fluid flow, facilitating pressure gradient formation, facilitating exudate drainage and the dispersion of growth factors and other medications. In some embodiments, the foam wound dressing material is configured to be expandable within a wound to form to the shape of the wound cavity, while in other embodiments, the foam wound dressing does not expand.

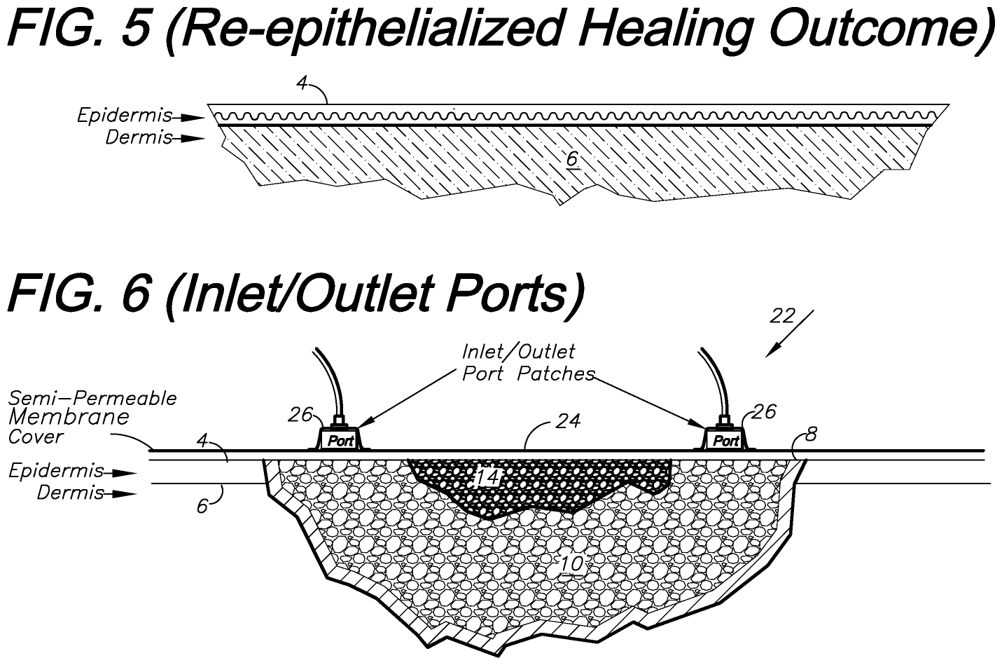

FIG. 5 shows a healing outcome with re-epitheliazation consisting of an intact epidermis over a dermis-like, healed granulation layer. The wound dressing 12 of the present invention can be modified, removed and reapplied as necessary to achieve such a healing outcome.

FIG. 6 shows a modified or alternative embodiment of the present invention comprising wound dressing 22 with a porous, semi-permeable membrane 24 covering the wound 2. Inlet/outlet port patches 26 can be placed where necessary and configured for extracting exudate or introducing solutions to the wound site. Such solutions can include growth factors, antibiotics and other medications. Pressure gradients can be formed by connecting the inlet/outlet port patches 26 to suction sources for outlet operating modes in NPWT applications, and to fluid delivery devices for inlet operating modes.

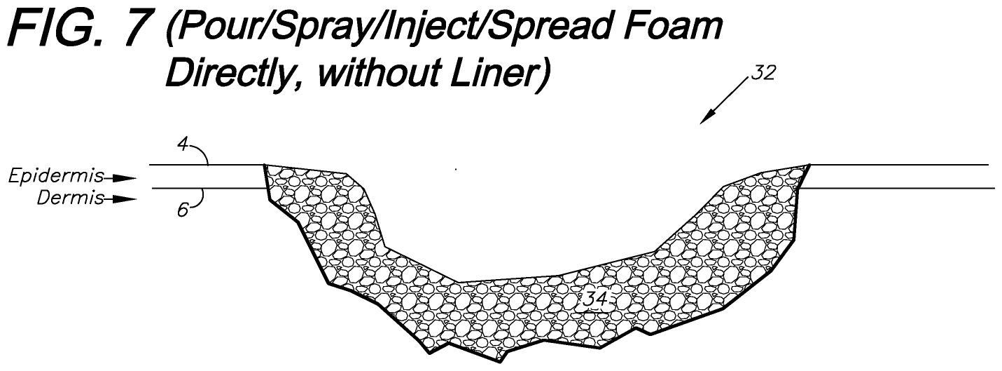

FIG. 7 shows yet another modified or alternative embodiment of the present invention comprising foam filler 34 poured, sprayed, injected or spread directly into the wound 2 without a liner. The volume of foam filler 34 applied to the wound 2 is variable. Pouring, spraying, injecting or spreading foam filler 34 in a liquid or semi-viscous state into the bed of the wound 2 enables controlling variables such as thickness, volume, evaporation and fluid transfer functions.

The foam wound dressing material can be configured to cure via a chemical curing agent, a photo-initiator curing agent, water moisture, or change in temperature. Different embodiments of the wound dressing material may be made up of a polyurethane ester, a polyurethane ether, a polyethylene glycol, a polyvinyl alcohol, a polylactic acid, a polyester, a polycaprolactone (PCL), a silicone-based derivative or a polysaccharide. Furthermore, the foam wound dressing may be formed by covalent bonds, ionic bonds, or hydrogen bonds.

The wound dressing of the present invention may be used with additional wound dressing and/or wound therapies, as desired. The dressing may further be covered with an adhesive dressing covering. Additionally, negative pressure or positive pressure may be applied to the wound and dressing. In some embodiments, the wound dressing is configured for compacting or compressing under negative pressure, while in other embodiments, the wound dressing is configured to hold its structure under negative pressure. As the wound heals, the wound dressing of the present invention can be configured for removal from the wound or the dressing material may be configured for being resorbed in the wound.

In embodiments incorporating a sacrificial component, a sacrificial solution may be dissolved into the wound dressing compound. Alternatively, a sacrificial solution can be dissolved into a solution and then added to the wound dressing compound system. Moreover, in other embodiments, the sacrificial solution is dissolved into a solution and added into the wound simultaneously with the residual foam component system. The sacrificial component may also be removed by the application of negative pressure or vacuum after its dissolution, or the sacrificial component may be dissolved into the wound site and taken up by the surrounding tissue.

In some embodiments of the present invention, the porous fraction of the foam insert can be modified by modulating molecular characteristics of the sacrificial porogen, including but not limited to modification of the molecular weight or size of the porogen. The porous fraction of the foam insert can alternatively be modified by modulating molecular characteristics of the residual foam component, including but not limited to modification of the molecular weight or size of the residual compound or modification of the relative concentration of the residual foam compound. In additional embodiments, the porous fraction of the foam is created by using gas as a porogen. The gas porogen may be dissolved, mixed or incorporated into the multi-solution system before application and allowed to dissolve, permeate or evaporate out of the foam upon application to the wound or the gas may be applied to the wound site promoting the formation of bubbles within the residual polymer foam as it cures. In some embodiments, the sacrificial porogen may be resorbed or dissolved within the wound environment or degraded and removed by enzymatic activity. In other embodiments, the sacrificial porogen is dissolved within the wound environment after a change in temperature or after application of a solvent over the foam material. The solvent may be aqueous-based, an acid, or a base and may or may not contain an enzyme.

In a preferred embodiment, the sacrificial porogen is a natural occurring biological compound, including but not limited a protein, polysaccharide, nucleic acid, or salt. Protein sacrificial porogens include but are not limited to collagen, gelatin, silk fibroin, and fibrin. Polysaccharide sacrificial porogens include but are not limited to dextran, xanthan gum, pectin, hyaluronic acid, carrageenan, guar gum, and cellulose. Polymer sacrificial porogens may also be used, including but not limited to polyethylene oxide, polyethylene glycol, polyvinyl alcohol, polyvinyl pyrrolidone, polyacrylic acid, polyacrylamides, polyphosphate, and hydroxypropyl methacrylamide.

A smaller, preformed foam material may further be used as a core element in conjunction with the multi-solution system of the present invention. This core foam insert may become a vacuum core under negative pressure.

In an exemplary embodiment, a synthetic polymer foam is utilized, preferably a polyurethane fabricated by mixing isocyanate and polyols, but alternative polymer foam materials may be used. A sacrificial porogen including natural and/or synthetic compounds that dissolve in water, such as polyethylene glycol or gelatin, or a gas contained within a solution is mixed with the foam material. In some embodiments, the sacrificial compound can also dissolve into the wound site naturally. Biological sacrificial components may include fibrin, collagen, and/or hyaluronic acid. Soluble bioactive factors utilized may include growth factors and/or exosomes. In an exemplary embodiment, a crosslinking or curing agent is applied to the foam material to cure the foam within the wound. The crosslinking or curing agent may be a natural enzyme or factor such as factor XIII or calcium, water, natural enzyme, biological agent, chemical agent, temperature change or a particular spectrum of light such as UV light.

In some embodiments, an amorphous gel liner is utilized either in conjunction with the foam dressing or standalone. The liner may be poured, sprayed, injected, or spread into the wound. In a preferred embodiment, the liner is a closed-cell material used in conjunction with an open-cell foam material superficial to or positioned closer to the surface than the liner. A liner may also be used alone with negative pressure therapy. In preferred embodiments, a closed-cell liner is made up of bioactive compounds or extracellular matrix (ECM), such as but not limited to fibrin, collagen, or hyaluronic acid. The liner may or may not need to be removed from the wound site during the wound healing process. Similarly, the foam dressing material also may or may not need to be removed from the wound site.

In an exemplary embodiment of a pourable foam dressing material, isocyanate is mixed with polyol and a sacrificial porogen of polyethylene glycol or gelatin to form the foam material. In an exemplary embodiment of a pourable gel liner material, a fibrinogen-based first solution containing factor XIII and bioactive growth compounds is mixed with a thrombin-based second solution containing calcium chloride and ECM compounds to form the liner material. The liner material may optionally further include a sacrificial porogen (if desired) and/or synthetic or natural polymer (to enhance structural stability of the final gel material).

### III. Examples of Exemplary Embodiments

The following is a non-limiting listing of additional exemplary embodiments of the present invention:
1. Fabrication of an open-cell foam from a pourable, sprayable, injectable, or spreadable multi-solution system that expands within a wound as it cures. This is achieved by utilizing a multi-component system of materials within the multi-solution system that will ultimately be used to compose foam solutions. One component is a residual foam insert created inside the wound cavity. Another component is a sacrificial component. The residual compound material can be fabricated from two or more separate solutions and may include a biological compound or a synthetic biomaterial. In an exemplary embodiment, the residual compound material is configured to remain within the wound bed. The sacrificial compound material can be a sacrificial porogen material. Preferably, the sacrificial compound material will be removed from the final foam by dissolution or absorption.
2. The foam wound insert in example 1, wherein the multi-solution system of the residual foam component creates an expanding foam upon mixture of solutions
3. The foam wound insert in example 1, wherein the multi-solution system of the residual foam component fills an entire wound without first needing to expand.
4. The foam wound insert in example 1, wherein the multi-solution system is premixed into a single chamber before pouring into a wound.
5. The foam wound insert in example 1, wherein the multi-solution system is mixed at the time of pouring multiple solutions into a wound.
6. The foam wound insert in example 1, wherein the multi-solution system is applied via a multi-syringe chamber system that mixes solution through a cannula tip immediately before application into a wound.
7. The foam wound insert in example 1, wherein the multi-solution system is poured into a wound and allowed to expand and cure.
8. The foam wound insert in example 1, wherein the multi-solution system is sprayed into a wound and allowed to expand and cure.
9. The foam wound insert in example 1, wherein the multi-solution system is injected into a wound and allowed to expand and cure.
10. The foam wound insert in example 1, wherein the multi-solution system is spread into a wound and allowed to expand and cure.
11. The foam wound insert in example 1, wherein the residual compound cures via a chemical curing agent.
12. The foam wound insert in example 1, wherein the residual compound cures via a photo-initiator curing agent.
13. The foam wound insert in example 1, wherein the residual compound cures via water moisture.
14. The foam wound insert in example 1, wherein the residual compound cures via temperature.
15. The foam wound insert in example 1, wherein the residual compound formed is a polyurethane ester.
16. The foam wound insert in example 1, wherein the residual compound formed is a polyurethane ether.
17. The foam wound insert in example 1, wherein the residual compound formed is a polyethylene glycol.
18. The foam wound insert in example 1, wherein the residual compound formed is a polyvinyl alcohol.
19. The foam wound insert in example 1, wherein the residual compound formed is a polylactic acid.
20. The foam wound insert in example 1, wherein the residual compound formed is a polyester, a polycaprolactone (PCL) or a silicon-based derivative
21. The foam wound insert in example 1, wherein the residual compound formed is a polysaccharide.
22. The foam wound insert in example 1, wherein the residual compound formed is a formed with peptide bonds.
23. The foam wound insert in example 1, wherein the residual compound formed is a formed with covalent bonds.
24. The foam wound insert in example 1, wherein the residual compound formed is a formed with ionic bonds.
25. The foam wound insert in example 1, wherein the residual compound formed is a formed with hydrogen bonds.
26. The foam wound insert in example 1, wherein the residual compound formed can be applied with additional wounds dressings.
27. The foam wound insert in example 1, wherein the residual compound formed is covered with an adhesive dressing.
28. The foam wound insert in example 1, wherein the residual compound formed is attached to a negative pressure system.
29. The foam wound insert in example 1, wherein the residual compound formed is attached to a positive pressure system, such as topical oxygen delivery and therapy.
30. The foam wound insert in example 1, wherein the residual compound formed will become compacted upon application of negative pressure.
31. The foam wound insert in example 1, wherein the residual compound can be removed from the wound as needed.
32. The foam wound insert in example 1, wherein the residual compound can be resorbed in the wound as needed
33. The foam wound insert in example 1, wherein the residual compound contains pores greater than 100 micrometers in size.
34. The foam wound insert in example 1, wherein the residual compound contains pores that are continuous.
35. The foam wound insert in example 1, wherein the residual compound is hydrophobic.
36. The foam wound insert in example 1, wherein the residual compound is hydrophilic.
37. The foam wound insert in example 1, wherein the relative concentration of sacrificial porogen to residual compound is changed to modify porosity.
38. The foam wound insert in example 1, wherein the sacrificial porogen is resorbed within the wound environment.
39. The foam wound insert in example 1, wherein the sacrificial porogen is dissolved within the wound environment.
40. The foam wound insert in example 1, wherein the sacrificial porogen is degraded/removed by enzymatic activity.
41. The foam wound insert in example 1, wherein the sacrificial porogen is dissolved within the wound environment by a change in temperature.
42. The foam wound insert in example 1, wherein the sacrificial porogen is dissolved by application of a solvent over the foam inert.
43. The solvent used to dissolve porogen in example 42, wherein the solvent is aqueous-based.
44. The solvent used to dissolve porogen in example 42, wherein the solvent is an acid.
45. The solvent used to dissolve porogen in example 42, wherein the solvent is a base.
46. The solvent used to dissolve porogen in example 42, wherein the solvent contains an enzyme.
47. The foam wound insert in example 1, wherein the sacrificial porogen used is a natural biological compound.
48. The natural biological compound in example 47, wherein the biological porogen is a protein.
49. The protein porogen in example 48, wherein the protein is collagen.
50. The protein porogen in example 48 wherein the protein is gelatin.
51. The protein porogen in example 48, wherein the protein is silk fibroin.
52. The protein porogen in example 48, wherein the protein is fibrin.
53. The natural biological compound in example 47, wherein the biological porogen is a polysaccharide.
54. The polysaccharide porogen in example 53, wherein the polysaccharide is dextran.
55. The polysaccharide porogen in example 53, wherein the polysaccharide is xanthan gum.
56. The polysaccharide porogen in example 53, wherein the polysaccharide is a pectin.
57. The polysaccharide porogen in example 53, wherein the polysaccharide is hyaluronic acid.
58. The polysaccharide porogen in example 53, wherein the polysaccharide is carrageenan.
59. The polysaccharide porogen in example 53, wherein the polysaccharide is guar gum.
60. The polysaccharide porogen in example 53, wherein the polysaccharide is cellulose.
61. The natural biological compound in example 47, wherein the biological porogen is a nucleic acid.
62. The foam wound insert in example 1, wherein the sacrificial porogen used is a salt.
63. The foam wound insert in example 1, wherein the sacrificial porogen used is a polymer.
64. The sacrificial porogen polymer in example 63, wherein the polymer is polyethylene oxide.
65. The sacrificial porogen polymer in example 63, wherein the polymer is polyethylene glycol.
66. The sacrificial porogen polymer in example 63, wherein the polymer is polyvinyl alcohol.
67. The sacrificial porogen polymer in example 63, wherein the polymer is polyvinyl pyrrolidone.
68. The sacrificial porogen polymer in example 63, wherein the polymer is polyacrylic acid.
69. The sacrificial porogen polymer in example 63, wherein the polymer is polyacrylamides.
70. The sacrificial porogen polymer in example 63, wherein the polymer is a polyphosphate.
71. The sacrificial porogen polymer in example 63, wherein the polymer is hydroxypropyl methacrylamide.
72. The foam wound insert system in example 1, wherein the sacrificial solution is dissolved into the residual foam compound system.
73. The foam wound insert system in example 1, wherein the sacrificial solution is dissolved into solution and then added the residual foam compound system.
74. The foam wound insert system in example 1, wherein the sacrificial solution is dissolved into solution and added simultaneously with the residual foam component system into the wound.
75. The foam wound insert system in example 1, wherein the sacrificial component is removed by negative pressure application under vacuum after its dissolution.
76. The foam wound insert system in example 1, wherein the sacrificial component is dissolved into the wound site and taken up by the tissue.
77. The foam wound insert system in example 1, wherein the porous fraction of the foam insert can be modified by modulating molecular characteristics of the sacrificial porogen.
78. The molecular characteristics in example 77, wherein the molecular weight or size of the porogen is modified.
79. The foam wound insert system in example 1, wherein the porous fraction of the foam insert can be modified by modulating molecular characteristics of the residual foam component.
80. The molecular characteristics in example 79, wherein the molecular weight or size of the residual compound is modified.
81. The molecular characteristics in example 79, wherein the relative concentration of the residual compound is modified.
82. The foam wound insert system in example 1, wherein the porous fraction of the foam is created by using gas as a porogen.
83. The gas porogen in example 82, wherein the gas is dissolved into the multi-solution system before application and allowed to dissolve out of the foam upon application to wound.
84. The gas porogen in example 82, wherein the gas is applied to the wound site promoting the formation of bubbles within the residual polymer foam as it cures.
85. The foam wound insert system in example 1, wherein a smaller preformed foam material is used as a core element in conjunction with the multi-solution system surrounding it.
86. The preformed foam material in example 85, wherein the core foam insert becomes the vacuum core under negative pressure.
87. Fabrication of an open-cell or closed-cell gel or foam that lines a wound from an initial pourable, sprayable, injectable, or spreadable multi-solution system that cures within the wound as it contours to the shape of the wound. The discontinuity, minimal pore size and low bulk porosity found with a closed-cell liner will decrease the rate at which tissue enmeshing occurs normally found with only using open-cell foam insert. However, the closed-cell liner is also porous enough to allow for fluid and exudate to be removed. The liner can be achieved in a similar fashion as describe in example 1, wherein a multi-solution system is used to apply to a wound which then covers the wound with or without the use of an expanding foam wound insert. When used with a foam wound insert, the gel and/or foam liner can be separate or detached from the foam insert or bound to the foam insert via the manipulation of the curation process or other methods, such as chemical or protein interactions between the liner and the insert. The liner can be fabricated with biological compounds or synthetic biomaterials. The liner can augment wound healing with the addition of bioactive factors or using a bioactive compound to construct the liner.
88. The gel and/or foam liner in example 87, wherein the liner is open-celled with continuous pore with diameters greater than 100 micrometers.
89. The gel and/or foam liner in example 87, wherein the liner is closed-celled with discontinuous pore with diameters less than 100 micrometers.
90. The gel and/or foam liner in example 87, wherein the liner is poured into a wound and allowed to cure.
91. The gel and/or foam liner in example 87, wherein the liner is cured via a photo-initiator.
92. The gel and/or foam liner in example 87, wherein the liner is cured via natural crosslinking compounds, which include minerals and/or ions.
93. The gel and/or foam liner in example 87, wherein the liner is cured with a chemical additive, which includes an acid, base, organic compound, or inorganic compound.
94. The gel and/or foam liner in example 87, wherein the liner is cured with water moisture.
95. The gel and/or foam liner in example 87, wherein the liner is cured with a change in temperature.
96. The gel and/or foam liner in example 87, wherein the liner is used to coat a preformed foam insert before application into a wound site.
97. The gel and/or foam liner in example 87, wherein the liner is used independently without other wound therapies.
98. The gel and/or foam liner in example 87, wherein the liner is used independently without other pharmacologic therapies.
99. The gel and/or foam liner in example 87, wherein the liner is used with a foam insert on top of the liner.
100. The foam insert in example 87, wherein the foam insert is open-celled.
101. The foam insert in example 87, wherein the foam insert is expandable and poured on top of the liner.
102. The gel and/or foam liner in example 87, wherein the liner is used with other pharmacologic therapies.
103. The gel and/or foam liner in example 87, wherein the liner is used with other wound dressings.
104. The gel and/or foam liner in example 87, wherein multiple liner layers are used.
105. The multilayered liner system in example 104, wherein one of the layers contains bioactive compounds.
106. The multilayered liner system in example 104, wherein at least one of the layers is a closed-cell liner.
107. The gel and/or foam liner in example 87, wherein the liner is used with negative pressure therapy.
108. The gel and/or foam liner in example 87, wherein the liner is poured into a wound site before application of an open-celled foam insert.
109. The gel and/or foam liner in example 87, wherein a liner solution system is poured into a wound and before complete curation a second solution system for an expanding foam insert is applied in order to bind the two systems.
110. The gel and/or foam liner in example 87, wherein the liner compound is hydrophobic.
111. The gel and/or foam liner in example 87, wherein the liner compound is hydrophilic.
112. The gel and/or foam liner in example 87, wherein the liner compound is biodegradable.
113. The gel and/or foam liner in example 87, wherein the liner compound is resorbable.
114. The gel and/or foam liner in example 87, wherein the liner is a polymer.
115. The polymer liner in example 114, wherein the polymer is a polyurethane ester.
116. The polymer liner in example 114, wherein the polymer is a polyurethane ether.
117. The polymer liner in example 114, wherein the polymer is a polyvinyl alcohol.
118. The polymer liner in example 114, wherein the polymer is a polylactic acid.
119. The polymer liner in example 114, wherein the polymer is a polyglycolic acid.
120. The polymer liner in example 114, wherein the polymer is a polycaprolactone.
121. The polymer liner in example 114, wherein the polymer is a polyester.
122. The gel and/or foam liner in example 87, wherein the liner compound is a natural biological compound.
123. The natural biological compound in example 122, wherein the biological compound is a protein.
124. The protein liner in example 123, wherein the protein contains collagen.
125. The protein liner in example 123, wherein the protein contains fibrin.
126. The protein liner in example 123, wherein the protein contains vitronectin.
127. The protein liner in example 123, wherein the protein contains elastin.
128. The protein liner in example 123, wherein the protein contains laminin.
129. The protein liner in example 123, wherein the protein contains thrombin.
130. The protein liner in example 123, wherein the protein contains silk fibroin.
131. The protein liner in example 123, wherein the protein contains gelatin.
132. The natural biological compound in example 122, wherein the biological compound is polysaccharide.
133. The polysaccharide liner in example 132, wherein the polysaccharide contains pectin.
134. The polysaccharide liner in example 132, wherein the polysaccharide contains hyaluronic acid.
135. The polysaccharide liner in example 132, wherein the polysaccharide contains cellulose.
136. The polysaccharide liner in example 132, wherein the polysaccharide contains chitosan.
137. The polysaccharide liner in example 132, wherein the polysaccharide contains keratan sulfate.
138. The polysaccharide liner in example 132, wherein the polysaccharide contains chondroitin sulfate.
139. The polysaccharide liner in example 132, wherein the polysaccharide contains dermatan sulfate.
140. The polysaccharide liner in example 132, wherein the polysaccharide contains heparin.
141. The gel and/or foam liner in example 87, wherein the liner solution system has soluble bioactive factors incorporated into the solutions.
142. The soluble bioactive factors in example 141, wherein the bioactive factors are growth factors.
143. The soluble bioactive factors in example 141, wherein the bioactive factors are enzymes.
144. The soluble bioactive factors in example 141, wherein the bioactive factors are cytokines 145. The soluble bioactive factors in example 141, wherein the bioactive factors are chemokines.
146. The soluble bioactive factors in example 141, wherein the bioactive factors are exosomes.
147. The soluble bioactive factors in example 141, wherein the bioactive factors are antimicrobial agents.
148. The soluble bioactive factors in example 141, wherein the bioactive factors are pharmacological agents.
149. The soluble bioactive factors in example 141, wherein the bioactive factors are MicroRNAs.
150. The soluble bioactive factors in example 141, wherein the bioactive factors are oligonucleotides.
151. The soluble bioactive factors in example 141, wherein the bioactive factors are covalently bound to the liner compound.
152. The soluble bioactive factors in example 141, wherein the bioactive factors are released about dissolution in the wound environment.
153. The soluble bioactive factors in example 141, wherein the bioactive factors are released upon degradation of the liner.
154. The soluble bioactive factors in example 141, wherein the bioactive factors are released by enzymatic activity.
155. The gel and/or foam liner in example 87, wherein the liner solution system has stem cells incorporated into the solutions.
156. The gel and/or foam liner in example 87, wherein the liner solution system has keratinocytes incorporated into the solutions.
157. The gel and/or foam liner in example 87, wherein the liner solution system has fibroblasts incorporated into the solutions.
158. The gel and/or foam liner in example 87, wherein the liner solution system has endothelial cells incorporated into the solutions.
159. The gel and/or foam liner in example 87, wherein the liner solution system has pericytes incorporated into the solutions.
160. The gel and/or foam liner in example 87, wherein the liner solution system has a combination of stem cells, keratinocytes, fibroblasts, endothelial cells, and/or pericytes incorporated into the solutions.
161. The gel and/or foam liner in example 87, wherein the liner is removed to apply a new liner as needed.
162. The gel and/or foam liner in example 87, wherein the liner is left inside the wound.
163. The gel and/or foam liner in example 87, wherein the liner degrades within the wound with use.
164. The gel and/or foam liner in example 87, wherein the liner resorbs within the wound with use.
165. The gel and/or foam liner in example 87, wherein the liner is biodegradable and used with a non-degradable liner superficial to it.
166. The gel and/or foam liner in example 87, wherein the liner is biodegradable and is used with a foam open-cell wound insert.
167. The foam insert in example 166, wherein the foam insert used poured into the wound on top of the liner and replaced as needed.
168. The gel and/or foam liner in example 87, wherein the liner is able to contract the wound under negative pressure therapy.
169. The gel and/or foam liner in example 87, wherein the liner is used preoperatively to prep a surgical site.
170. The gel and/or foam liner in example 87, wherein the liner is used to intraoperatively to improve surgical outcomes.
171. The gel and/or foam liner in example 87, wherein the liner is used postoperatively to improve surgical outcomes.
172. The gel and/or foam liner in example 87, wherein the liner is used to attach or bind other dressings to a wound.
173. The foam wound insert in example 1, wherein the wound insert is used preoperatively to prep a surgical site.
174. The foam wound insert in example 1, wherein the wound insert is used to intraoperatively to improve surgical outcomes.
175. The foam wound insert in example 1, wherein the wound insert is used postoperatively to improve surgical outcomes.
176. The gel and/or foam liner in any of the above properties, wherein the liner solutions are sprayed into a wound instead of poured.
177. The gel and/or foam liner in any of the above properties, wherein the liner solutions are injected into a wound instead of poured.
178. The gel and/or foam liner in any of the above properties, wherein the liner solutions are spread into a wound instead of poured.
179. The foam wound insert in example 1, may be structurally manipulated via use of an electromagnetic field prior to curing.
180. The foam wound insert in example 1, may be structurally manipulated via use of an electromagnetic field post curing.
181. The foam wound insert in example 1, may be used to deliver an electrical current to stimulate cell proliferation and migration within the wound site.
182. The foam wound insert in example 1, may be structurally manipulated via use of an osmotic gradient prior to curing.
183. The foam wound insert in example 1, may be structurally manipulated via use of an osmotic gradient post curing.
184. The foam wound insert in example 1, may be used to change/control temperature within the wound site.
185. The gel and/or foam liner in example 87, may be structurally manipulated via use of an electromagnetic field prior to curing.
186. The gel and/or foam liner in example 87, may be structurally manipulated via use of an electromagnetic field post curing.
187. The gel and/or foam liner in example 87, may be structurally manipulated via use of an osmotic gradient prior to curing.
188. The gel and/or foam liner in example 87, may be structurally manipulated via use of an osmotic gradient post curing.
189. The gel and/or foam liner in example 87, may be used to change/control temperature within the wound site.
190. The gel and/or foam liner in example 87 or foam wound insert in example 1, wherein the multi-solution device system of an injectable material can be utilized in situations where separated bone, ligaments, tendons, and/or tissues are repaired via application of an injection of a glue-type fixating device, a spacer joining the two segments, or even simulating a plate by applying linearly across the tissue in case of a fracture, rupture, or tear. Utilization of an injectable material that can structurally stabilize in situ and support and stabilize a closed wound fracture, rupture, or tear of tissue that provides weight bearing support for the body without requiring open surgical correction.
191. The injectable device in example 190, wherein the solutions are applied via syringe injection.
192. The injectable device in example 190, wherein the solutions are applied via a percutaneous catheter or tubing.
193. The injectable device in example 190, wherein the solutions are applied via syringe injection.
194. The injectable device in example 190, wherein the device is able to fix tissue in place.
195. The injectable device in example 190, wherein the device is used in conjunction with an external fixation device such as a cast or a splint.
196. The injectable device in example 190, wherein the device is applied to tissue and provides structural support.
197. The injectable device in example 190, wherein the device is used to bind prosthetic devices in the body.
198. The injectable device in example 190, wherein the device is used to coat a joint articulation.
199. The injectable device in example 190, wherein the device is used as a spacer to join two or more segments of tissue.
200. The injectable device in example 190, wherein the device is applied after negative pressure application through a tubing or catheter to drain the interior of a closed wound.
201. The injectable device in example 190, wherein the device is used with negative pressure after device insertion to promote healing
202. The injectable device in example 190, wherein the device is biodegradable and resorbed by the body over time
203. The injectable device in example 190, wherein the device does not degrade and may need to be removed at a later point in time.
204. The removal of the injectable device in example 203, wherein the device is removed via an open surgery through an incision.
205. The removal of the injectable device in example 203, wherein the device is removed via a percutaneous port.
206. The removal of the injectable device in example 203, wherein the device is removed by dissolution into a solution and evacuated from the wound site of implantation via negative or vacuum pressure.

It is to be understood that the invention can be embodied in various forms and is not to be limited to the examples specifically discussed above. The range of components and configurations which can be utilized in the practice of the present invention is virtually unlimited.

Having thus described the invention, what is claimed as new and desired to be secured by Letters Patent is:

1. A wound treatment system comprising:
a foam dressing configured for application to and for lining or filling a wound cavity;
said foam dressing being comprised of a foam mixture of a first component and a second component;
said foam mixture is configured for curing within said wound cavity; and
wherein said foam dressing is configured for compacting and for accommodating transfer of fluid from said wound out of said wound under negative or other pressure gradient system.

2. The wound treatment system according to claim one wherein said foam mixture is configured for curing in response to application of a curing agent.

3. The wound treatment system according to claim 1, wherein:
said foam dressing further comprises a third component; and
said third component comprises a sacrificial porogen.

4. The wound treatment system according to claim 1, wherein:
said foam dressing comprises an open-cell foam.

5. The wound treatment system according to claim 1, which includes:
a foam wound liner lining said wound bed; and
a foam wound filler poured, sprayed, injected or spread in said liner.

6. The wound treatment system according to claim 1, which includes one of a hydrogel and a fibrin-based glue in said foam.

7. The wound treatment system according to claim 1, which includes:
a semi-permeable membrane covering said foam mixture; and
at least one of: an inlet port connected to said membrane and a fluid source; and an outlet port connected to said membrane and a suction device, said outlet port configured for discharging exudate from said wound via said outlet port.

8. The wound treatment system according to claim 6, wherein said foam wound liner and said, foam wound filler comprise foam materials with different cellular and pore configurations.

9. The wound treatment system according to claim 1, which includes:
a bioactive synthetic or natural compound applied to said foam material and configured for promoting tissue growth, cell migration, proliferation, improving dimensional stability or augmenting porous fractions.

10. The wound treatment system according to claim 1, further comprising:
an amorphous wound liner configured for application to and for conforming to said wound cavity;
said wound liner being comprised of a mixture of a first liner component and a second liner component;
wherein said wound liner is configured for curing within said wound cavity in response to application of a liner curing agent; and
wherein said wound liner is configured for application to said wound in a deeper position in relation to said foam dressing.

11. The wound treatment system according to claim 1, which includes:
a foam insert embedded in said foam filler;
pore size and bulk porosity variables of said foam effect compactability or compressibility of said foam insert; and
said compactability or compressibility of said foam insert effect degrees of physiochemical and mechanotransduction response of damaged tissue in said wound site.

12. The wound treatment system according to claim 3 wherein:
said third liner component comprises a liner bioactive agent.

13. The wound treatment system according to claim 12, wherein:
said liner bioactive agent is configured for release from said wound liner into said wound cavity.

14. The wound treatment system according to claim 3, wherein:
said wound liner further comprises a fourth liner component;
said third liner component comprises a liner sacrificial porogen; and
said fourth liner component comprises a liner bioactive agent.

15. The wound treatment system according to claim 4, wherein:
said wound liner comprises a closed-cell foam.

16. A method of treating a wound comprising the steps of:
mixing a first component and a second component forming an amorphous foam dressing;
applying said foam dressing into a wound cavity;
said foam dressing forming to and filling said wound cavity;
applying a curing agent to said wound; and
said foam dressing curing within said wound cavity.

17. The method according to claim 11, further comprising the steps of:
applying negative or other pressure gradient to said wound;
said foam dressing compacting under said negative or other pressure gradient; and
fluid from said wound transferring from said wound through said foam dressing and out of said wound under a pressure gradient.

18. The method according to claim 11, further comprising the steps of:
mixing a first liner component and a second liner component forming an amorphous wound liner;
applying said wound liner into said wound cavity; and
wherein said applying said foam dressing into said wound cavity comprises applying said foam dressing into said wound cavity closer to the surface than said wound liner.

19. The method according to claim 11, wherein:
said mixing said first component and said second component forming said foam dressing further comprises mixing a third component with said first and second components; and
said third component comprises a sacrificial porogen.

20. The method according to claim 14, further comprising the step of:
removing said sacrificial porogen from said foam dressing.
